(19) United States
(12) Patent Application Publication
(10) Pub. No.: US 2024/0020708 A1
(43) Pub. Date: Jan. 18, 2024

(54) CARBON OFFSET PLATFORM

(71) Applicant: Carbon Metrics Global, Dayton, OH (US)

(72) Inventor: Aurea Rivera, Dayton, OH (US)

(21) Appl. No.: 18/354,564

(22) Filed: Jul. 18, 2023

Related U.S. Application Data
(60) Provisional application No. 63/390,175, filed on Jul. 18, 2022.

(51) Int. Cl. G06Q 30/018; G01N 33/24

(52) U.S. Cl. CPC G06Q 30/018; G01N 33/24

(57) ABSTRACT

A platform is provided for evaluating carbon sequestration, e.g., in the form of carbon offsets. The platform can generate standards for carbon offsets that are trustworthy and verifiable. In particular, the platform carbon evaluation is predicated upon verification and validation attributes that include additionality, permanence, leakage, or combinations thereof.

### CROSS REFERENCE TO RELATED APPLICATIONS

This application claims the benefit of U.S. Provisional Patent Application Ser. No. 63/390,175, filed Jul. 18, 2022, having the title “CARBON OFFSET PLATFORM”, the disclosure of which is hereby incorporated by reference.

### BACKGROUND

The present disclosure relates in general to carbon offsets, and in particular, to a platform, method, and standard(s) for the measurement, reporting and verification of carbon, e.g., in soil.

Carbon offsets reduce, remove or avoid greenhouse gas (e.g., carbon dioxide) emissions. Moreover, in some applications, carbon offsets can be expressed as “carbon credits” that can be exchanged in a carbon market. Basically, a buyer, such as a corporation that generates carbon emissions can purchase carbon credits as a way to offset a total carbon footprint.

### BRIEF SUMMARY

According to aspects of the present invention, a carbon offset platform is provided. The carbon offset platform comprises a database and a processing system. The database stores electronic geospatial-specific data, where distinct bounded geographic regions can be associated with the stored electronic geospatial-specific data. The processing system measures carbon sequestration using a carbon model that factors additionality, permanence, leakage, or a combination thereof. The processing system is further operatively programmed to receive and process at least one sample collected from within the bounded geographic region to determine the amount of carbon sequestered thereby. The processing system is further operatively programmed to evaluate the collected and processed sample data, a reference baseline, the stored electronic geospatial-specific data, and the model to derive a carbon sequestration stock signature to carbon credit translation. The processing system is still further operatively programmed to provide an output.

### BRIEF DESCRIPTION OF THE SEVERAL VIEWS OF THE DRAWINGS

FIG. 1 is a block diagram of a carbon offset platform according to aspects of the present disclosure;

FIG. 2 is a flow chart illustrating a process for generating a carbon sequestration estimation, according to aspects of the present disclosure;

FIG. 3 is a block diagram of a computer platform that can be utilized to implement aspects of the present disclosure; and

FIG. 4 is a block diagram of a carbon offset system, according to aspects of the present disclosure.

### DETAILED DESCRIPTION

The release of greenhouse gases, such as the release of carbon dioxide into the atmosphere caused by industrialization (anthropogenic), can be linked to climate change. As such, there is an emerging and current climate challenge for our country to be net-zero in carbon (greenhouse gas) emissions by the year 2050. Net-zero refers to a condition where efforts are placed to offset or otherwise balance carbon emissions from businesses with projects that remove carbon dioxide from the atmosphere. One key source to remove carbon dioxide from the atmosphere is in the agriculture industry. Since plants, trees, and vegetation can capture carbon, there is a recognition that incentivizing farmers to use cover crops and performing other soil conservation practices can lead to the removal of carbon dioxide from the atmosphere. This realization has led to the commoditization of carbon offsets in the form of carbon credits that can be bought, sold, or traded.

Notably, agriculture in the form of farming activity can contribute to the release of greenhouse gases. However, agriculture also provides an avenue for mitigating their carbon footprint and capturing carbon from the atmosphere in what is referred to as a “carbon sequestration mode” (storing carbon in vegetation and soils). A recent United States Department of Agriculture census identified over 1 billion acres in the United States that was tagged as being used for farming. Of the 1 billion acres, 140 million acres were actively engaged in conservation activities like no-till and cover crops, with less than 3% of these acres enrolled in a voluntary carbon market program. As such, the agricultural community represents a large resource to capture carbon from the atmosphere.

System Overview

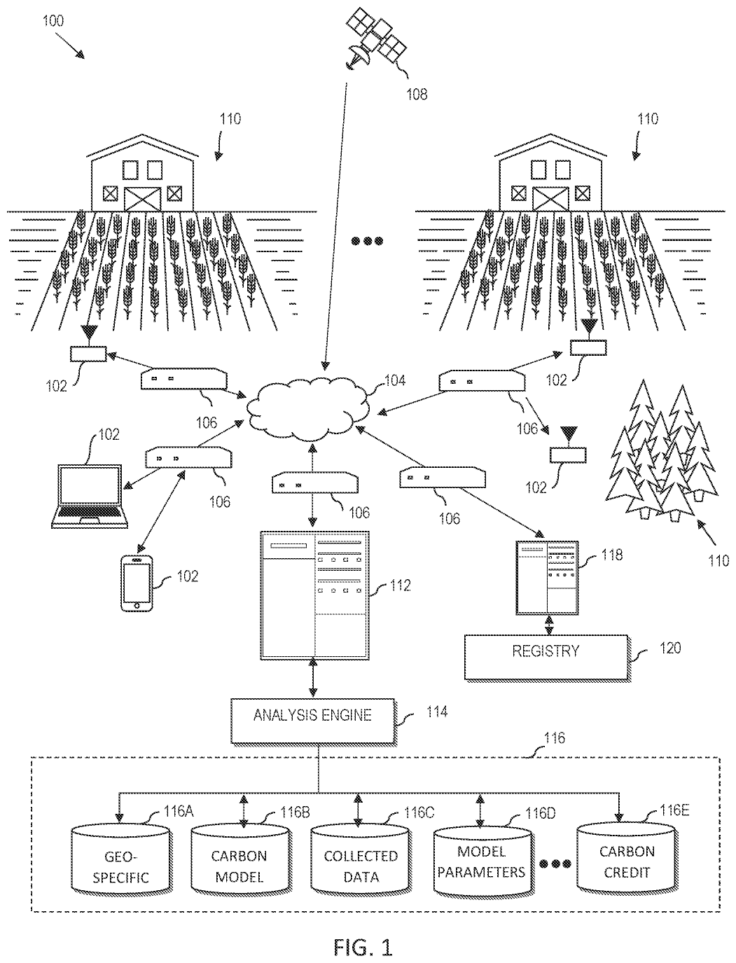

Referring now to the drawings and in particular to FIG. 1, a general diagram of a computer system 100 is illustrated according to various aspects of the present disclosure. The illustrated computer system 100 is a special purpose (particular) system that operates using geospatial-based features, event/data descriptions, collected data, machine learning, artificial intelligence, data analysis, or combinations thereof, to perform, among other activities, a carbon sequestration stock signature to a carbon credit measurement conversion. Additional processes can include for example, reporting and verification of carbon, e.g., in soil, auditing, transaction processing, combinations thereof, examples of which are described in greater detail herein.

The computer system 100 comprises a plurality of hardware processing devices designated generally by the reference 102 that are linked together by one or more network(s), which are designated generally by the reference 104.

The network(s) 104 provides communications links between the various processing devices 102 and may be supported by networking components 106 that interconnect the processing devices 102, including for example, routers, hubs, firewalls, network interfaces, wired or wireless communications links and corresponding interconnections, cellular stations and corresponding cellular conversion technologies (e.g., to convert between cellular (e.g., 5G) and Transmission Control Protocol/Internet Protocol methodologies (TCP/IP), etc.). Moreover, the network(s) 104 may comprise connections using one or more intranets, extranets, local area networks (LAN), wide area networks (WAN), wireless networks (Wi-Fi), the Internet, including the world wide web, cellular and/or other arrangements for enabling communication between the processing devices 102, in either real time or otherwise (e.g., via time shifting, batch processing, etc.).

A processing device 102 can be any device capable of communicating over the network 104. Examples of processing devices 102 include a transactional system, purpose-driven appliance such as a smart sensor, personal data assistant (PDA), palm computers, cellular devices including cellular mobile telephones and smart telephones, tablet computers, netbook computers, notebook computers, personal computers and servers.

The illustrative system 100 can leverage geospatial-specific data, as will be described in greater detail herein. In this regard, geospatial-specific data can come from any number of sources, such as remote sensing satellite(s), payloads, unmanned aerial systems (UAS) payloads, platform imagery across the electromagnetic spectrum, collected from one or more satellites, from third-party servers that have previously collected such geospatial-specific data, etc. For sake of clarity of discussion, the collection of geospatial-specific data is schematically represented by satellite 108. In some embodiments, the collected geospatial-specific data, e.g., geospatial-based images include terrestrial frequency responses of land 110, e.g., agricultural areas such as farmland, forested areas, fields, plant-rich environments, etc. In practice, there can be multiple satellites 108, multiple geo-spatial data gathering payloads, maps, surveys, combinations thereof, etc.

In some embodiments, the geospatial-specific data can be associated with distinct, bounded geographical regions, e.g., tracts of land 110, such as agricultural areas including farmland, forested areas of land, plant-rich areas of land, other property, etc., that can be uniquely identified to an owner. In this regard, the geographical regions represent real/natural bounded regions of our physical planet. The bounded regions can be further defined or otherwise limited in size and/or boundary definition based upon human ownership, where a person/individual or group, business, or government is the owner (e.g., holder of a title and/or deed to a bounded region of land) by way of example.

The illustrative computer system 100 also includes a processing device implemented as a server 112 (e.g., a web server, file, data storage servers, and/or other data and user authentication processing devices) that supports a carbon sequestration stock signature measurement analysis engine 114 and corresponding data sources (collectively identified as data sources 116). The analysis engine 114 and data sources 116 provide the resources to implement and store the carbon sequestration measurement, carbon sequestration stock signature to carbon credit translation, reporting and verification of carbon, e.g., in soil, as described in greater detail herein.

In an exemplary implementation, the data sources 116 are implemented by a collection of databases that store various types of information related to carbon emissions, sequestration, geographic location, soil type, conservation practices attributed to that location, historical weather/climate measurements, historical geospatial data collections, value information, analytical data, combinations thereof, etc.

However, these data sources 116 need not be co-located. Additionally, the data sources 116 need not be co-owned/managed. In the illustrative example, the data sources 116 include proprietary data that supports the platform processing, remote data sources that supply information to the platform implemented by the analysis engine 114, data about sources of emitting carbon, data about sources of sequestering carbon, combinations thereof, etc.

Additionally, in an example embodiment, one or more processing devices, such as a server computer, is designated as a trusted authority 118. The trusted authority 118 supports a Carbon Credit Registry 120, examples of which are described in greater detail herein.

Example Platform

Unfortunately, current conventional carbon sequestration approaches cannot adequately provide trusted estimates that measure, quantify, monitor, report, and verify carbon sequestration in soil such as used in agricultural applications. For example, soil modeling represents a way to estimate the ability of a soil region to contain carbon. However, existing carbon quantification models have significant drawbacks. For instance, existing carbon quantification models lack a standardized method for measuring and quantifying carbon sequestration holdings that address key carbon credits' attributes. Additionally, existing carbon quantification models require permanent access to agronomic data holdings associated with farm operations.

Yet further, existing carbon quantification models fail to quantify certain verification and validation attributes that may be sufficient to establish a trusted carbon credit measure.

However, according to aspects herein, the analysis engine 114 implements a platform for measuring carbon sequestration, which is predicated upon verification and validation attributes that include additionality, permanence, leakage, or combinations thereof.

### Verification and Validation Attribute Definitions

As used herein:

Additionality—refers to the greenhouse gas mitigation that would not have occurred without the acquisition of a carbon offset. Participants do not generally earn offset credits for continuing existing practices and activities but rather for initiating new methods and activities.

Permanence—describes the issue of ensuring the removal of carbon dioxide from the atmosphere is permanent and not reversed at a future point in time. In some embodiments, permanence refers to the duration of the stored carbon. For instance, an example carbon standard may require 100-year permanence, or some other reasonable measure of time.

Leakage—refers to an increase in greenhouse gas emissions outside of a project area in response to decreases in production within the project area. High-quality carbon offsets are generated with processes that take steps to prevent leakage. In some embodiments, carbon leakage occurs when an emissions-reduction policy such as carbon price inadvertently causes an increase in emissions in other jurisdictions that do not have equivalent emission-reduction policies.

### Additional Attribute Definitions

Realness—refers to whether an offset represents an actual and quantifiable amount of carbon sequestration or reduction in greenhouse gas emissions.

Verification—ensures that the offsets were quantified correctly. For instance, a verification can comprise a process usually conducted by a third party.

Carbon sequestration “quality” is a metric. As long as buyers of agriculture credits perceive differences in the quality of credits generated through alternative protocols, initiatives generating high-quality credits will gain market share. In contrast, economically inferior quality carbon credits will exit the market. The latter represents a systemic risk for farmers and credit buyers. The normalization of farming practices and quantifiable carbon metrics signatures mitigate the risk to farmers, enabling a trusted carbon market to include transferable partial and fill credits across protocols.

Platform Architecture

According to various aspects of the present disclosure, the analysis engine 114 implements a platform that utilizes machine learning, and ultimately, artificial intelligence to leverage available data with understandings of carbon removal from the atmosphere to derive a platform capable of analyzing, processing, and understanding carbon removal schemes. This carbon sequestration analytical architecture enables the platform to output carbon offset data, carbon credits, carbon sequestration verification, carbon sequestration validation, carbon offset auditing, carbon sequestration valuation, carbon sequestration education including attribution to methods, approaches and techniques to optimize or otherwise improve carbon sequestration for a given application (e.g., farmer, etc.). The carbon offset modeling and techniques for validation and verification further enable the creation of a carbon sequestration stock signature metric standard that can be independently verified.

Example Carbon Offset Platform

In an example embodiment, a carbon offset platform is implemented by the analysis engine 114 interacting with the data sources 116 and optionally, any combination of other resources described with reference to FIG. 1.

For instance, as illustrated, the data sources 116 can include by way of example, a first source 116A, e.g., a database, which stores electronic geospatial-specific data. For instance, distinct bounded geographic regions can be associated with the stored electronic geospatial-specific data in the database 116A.

A second source 116B defines a carbon model. Keeping with the examples herein, the model is constructed to factor additionality, permanence, leakage, or a combination thereof.

A third source 116C, e.g., a database, stores collected data, such as samples from within the bounded geographic regions. As described more fully herein, the collected data is utilized to determine the amount of carbon sequestered by the corresponded bounded geographic region. The third data source 116C can also store a reference baseline for the corresponding bounded geographic region.

A fourth source 116D, e.g., a database, stores model parameters, e.g., parameters for additionality, permanence, leakage, etc., which are used by the model at 116B. The fourth source 116 can also store, for example, agronomic data that comprises parameterized inquiries, collected facts, collected extrinsic information, effects on or caused by neighboring regions, historical information, or combinations thereof.

A fifth source 116E, e.g., a database, stores outputs, e.g., carbon credit information, the outputs from the model at 116B, etc.

A processing system, which can include server 112, measures carbon sequestration using a carbon model, e.g., the model at 116B, that factors additionality, permanence, leakage, or a combination thereof, e.g., from the model parameters at 116D.

The processing system is further operatively programmed to receive and process at least one sample, e.g., stored in the collected data 116C, from within the bounded geographic region defined by the geospatial-specific data stored at 116A, to determine the amount of carbon sequestered thereby.

The processing system is further operatively programmed to evaluate the collected and processed sample data at 116C, a reference baseline at 116C, the stored electronic geospatial-specific data at 116A, and the model at 116B (using model parameters at 116D) to derive a carbon sequestration stock signature to carbon credit translation, e.g., which is stored in the carbon credit output at 116E. This result is utilized to provide an output, as will be described in greater detail herein.

Standardization

Aspects of the present disclosure relate in general to carbon offsets, and in particular, to a platform, method and standards for the measurement, reporting and verification of carbon, e.g., in soil. In some embodiments, the platform analyzes, processes, standardizes, or combinations thereof, carbon offsets, e.g., for the agriculture industry.

Carbon offsets can be expressed as “carbon credits”. Carbon credits are certificates representing quantities of greenhouse gases kept out of the air or removed from the air. In an example embodiment, one carbon credit is equivalent to one metric ton of greenhouse gases removed from the atmosphere and sequestered in the soil.

An existing problem with current carbon credit systems is that there is no scientifically rigorous way to verify or certify that a quantity of carbon was actually removed from an area corresponding to the certificate (the trusted dilemma). However, as described more fully herein, some embodiments herein provide a carbon credit signature measurement.

In some embodiments, the platform harnesses the power of remote sensing techniques across the air and space domains that, when properly calibrated, processed, and analyzed, result in trusted carbon credit sequestration measurements. The carbon credit measurements described more fully herein, provide a trusted, minimally intrusive foundation for the climate-smart technologies industries.

The measurement, verification and validation modeling herein addresses critical attributes for an industry-standard trusted carbon credit: additionality, permanence, and leakage.

Example Platform

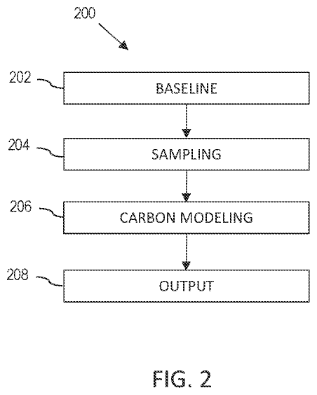

Referring to FIG. 2, a process 200 implements a carbon analysis, which can be implemented using the system 100 of FIG. 1. In this regard, reference to the “platform” can be implemented by the analysis engine 114 (FIG. 1) interacting with the various described components of the system 100.

According to aspects herein, the platform derives a reference baseline at 202. In example embodiments, the reference baseline can be derived from source data such as historical geospatial geo-rectified remote sensing image data of areas of land. In some embodiments, the reference baseline can be derived from geo spatial-based data, such as remote sensing satellite (or UAS) imagery (see for example, satellite 108, which collects images of the agricultural areas, e.g., farmland 110, FIG. 1). The image and/or other geospatial-based data forms a part of the carbon sequestration analysis.

In some embodiments, the reference baseline can be derived in part, from remote sensing geo-rectified geospatial image data that is processed to identify bounded regions that can be attributed to a single source of carbon sequestration pool credit, e.g., a farm field owned by an identifiable entity. In some embodiments, that bounded region can be further divided into features, e.g., “cells” or “areas” within the bounded region. Data analysis on the extracted features is utilized to ascertain parameters associated with the bounded region, e.g., size, use, amount of vegetation, carbon sequestration potential, whether the area is a field for planning crops, etc.

Carbon Metrics Baseline

In some embodiments, carbon sequestration parameters and agricultural conservation practices help establish the permanence of the carbon credit and the impact of the agricultural conservation practices on other sites. A reference baseline is useful to the establishment of carbon sequestration lower bound (infimum) that can be used to assess future gains in carbon sequestration. In some embodiments, the reference baseline quantifies a soil's organic carbon sequestration content at any given location and time (carbon sequestration spatial and temporal components). Without reference baseline measurements of captured soil organic carbon before implementing agricultural conservation practices, current carbon credits protocols do not permit agricultural producers' claims that carbon sequestration has taken place.

As such, according to aspects herein, a carbon baseline product line is provided. The baseline herein leverages an in-depth understanding of remote sensing techniques across the air and space domains, resulting in a temporal, geo-rectified carbon sequestration measurement. As such, aspects herein may only need to provide the conservation practices implemented at the site.

Sampling

The process 200 performs sampling at 204. For instance, a process may be implemented to receive and process actual (physical) soil samples, or other physical samples that are collected from a specified location. As an illustrative example, using the remote sensing image data collected in the reference baseline at 202, a geographical area can be parsed into adjacent cells or regions. One or more soil samples can be collected from the associated bounded region, e.g., one or more samples per cell. In some embodiments, one or more samples (statistically significant sample number determined based upon application) can be collected from one or more cells. In some embodiments, samples from every cell are not strictly required. Rather, in some embodiments, samples can be collected from some, but not necessarily all of the cells.

The collected soil samples are analyzed to determine the amount of carbon sequestered thereby (or a suitable soil feature proxy than can be used to estimate the carbon sequestered at the sample location). For instance, spectral analysis can be implemented to identify the frequency response of the sample. As a non-limiting example, a soil sample can be incinerated. A spectral analysis is performed, and the results are compared against calibrated carbon sequestration stock signature data, e.g., to detect emissions within a predetermined frequency and spectral bandwidth.

In some embodiments, sampling can be implemented by physical collection, which is sent to a laboratory for analysis. In some embodiments, sampling may be carried out using sensors placed at the location of interest. In some embodiments, the sensors are “smart sensors” that define a processing device 102 (see processing devices 102, which are illustrated directly below the farm fields in FIG. 1) capable of communicating across the network 104 back to the server 112 (FIG. 1).

In some embodiments, the sample collection process is periodic, e.g., yearly, bi-annually, three times a year, or any other timeframe determined to be relevant to the platform. The frequency of the collections will be determined by the agronomic life-cycle with a minimum of three collection events.

Carbon Metrics—Signatures

The accountability and trustworthiness of a carbon credit generated by the platform 200 can depend on the ability of the platform 200 to characterize carbon sequestration's permanence, leakage property, and the additionality of agricultural soil carbon sequestration activities. These attributes (permanence, leakage, additionality) relate to the integrity and consistency of using location-specific projects as an offset against greenhouse gas emissions generated in other sectors. In some embodiments, the net carbon benefits accounts for the fact that the sequestered carbon may be stored temporarily/impermanently, the project may displace emissions outside the project boundaries (leakage), and the project's carbon sequestration may not be entirely additional to what would have occurred anyway under business-as-usual (no project) conditions.

As such, according to aspects herein, additionality, permanence, and leakage attributes are addressed by conducting remote sensing collections, carbon sequestration stock signature calculations, verification, and validation. In an example embodiment, three (3) remote sensing measurements are collected in a farming calendar year. In the northern hemisphere these remote sensing measurements can be scheduled for example, at: the peak photosynthesis response cycle (typically in the middle of July); the end of the harvesting period (late September and October, location dependent), which will coincide with the cover crops planting period, and at the early stages of the planting season (middle of April to May). These three sets of carbon in the soil measurements permit the complete characterization of the nature of the carbon sequestration cycle at a given location (signature).

Modeling

The platform performs carbon modeling at 206. The modeling combines remote sensing data collection, actual sample data, and optionally other data to evaluate the ability of the bounded region, cell, location, etc., to sequester carbon. For instance, the model(s) can take into consideration, the reference baseline at 202, results of sampling at 204, combinations thereof, etc.

In some embodiments, the modeling can predict a carbon offset. An example implementation evaluates carbon offsets by evaluating additionality, permanence, leakage, or a combination thereof.

Carbon Metrics—Modeling and Simulation

Agriculture and forestry sectors can play an essential role in limiting greenhouse gases in the atmosphere. Conservation and land management practices can reduce emissions of carbon dioxide, methane, and nitrous oxide associated with crop and livestock production, increase the quantity of carbon stored in soils and above-ground vegetation and generate renewable fuels that recycle carbon dioxide from the atmosphere.

Climate Change policy advocates have stated an aspirational goal of reaching a net-zero carbon state by 2050. The US Department of Agriculture (USDA) land in farm estimates are 897 million acres; forests bring an additional 500 million acres. Complete carbon-sequestration characterization for the entire agricultural landmass is likely to be required to adequately capture the carbon sequestration metrics and, thus, advance towards the net-zero goal.

However, conventional accounting and mitigation tools and methods do not fully address the carbon metric market's accounting, verification, and validation requirements.

As such, in some embodiments herein, modeling and simulation data layers harness the power of an increasingly persistent remote sensing capability with advances in modeling and simulation to generate a trusted characterization of the agricultural landmass of the US. In some embodiments, the modeling can generate a complete carbon-sequestration characterization for the entire agricultural landmass of a defined area (e.g., the entire United States). The model(s) herein can leverage artificial intelligence and machine learning techniques that improve their forecasting ability as training data (acquired as part of the above reference baseline, carbon sequestration stock signature efforts) improves the accuracy of the carbon sequestration model.

Platform Output

The process 200 provides an output at 208. The output can comprise a verified carbon sequestration amount, carbon offset amount, etc., e.g., X metric tons per acre.

In some embodiments, the output is in the form of a carbon credit, share, certification, verification, validation, audit, or other output. In other embodiments, the system 100 of FIG. 1 and/or platform 200 of FIG. 2 can be utilized to certify, validate, and verify a carbon sequestration associated with a carbon offset.

In some embodiments, additional agronomic data is collected. Additional data can comprise parameterized inquiries (e.g., does the farmer plant cover crops, does the farmer practice no-till, etc.), collected facts (crops to be planted, historical soil data, etc.), collected extrinsic information (artifacts from a farmer implementing carbon sequestration, effects on or caused by neighboring regions, historical information, e.g., climate data, etc.).

The additional collected data can be used to derive additionality, permanence, leakage, etc.

In an example embodiment, the process 200 generates a carbon content baseline using satellite remote sensing data and optionally, data with regard to a specific farm solution. The process can integrate unmanned aerial systems (UAS) with the appropriate sensor architecture. The result is an end-to-end, fully documented, scientifically driven trusted carbon credit metric estimate.

Attribution and Feedback to Farmers

A significant number of agricultural producers do not currently use farm-level data software. In this regard, in some embodiments, the platform herein (e.g., via the output 208) is adapted to expose farmers to the tools they need to auto collect and manage robust data about their farming and agricultural conservation practices with as little burden as possible so they can get the most out of every digital acre for profitability and/or sustainability. The platform herein provides an effective way to produce trusted carbon measurements. Moreover, aspects of the present disclosure provide a platform that functions as a carbon sequestration metric provider for the climate-smart commodity market (any agricultural commodity that is produced using agricultural (farming, ranching, or forestry) practices that reduce greenhouse gas emissions or sequester carbon).

Carbon Metrics—Farming Carbon Metrics

As the carbon sequestration effort advances, embodiments herein provide agricultural producers with a non-intrusive way to characterize their carbon credit holdings. Access to their carbon credit information provides marketing opportunities resulting in higher carbon credit prices.

As such, according to aspects herein, a farming subscription can be implemented to provide the agricultural producer with a verified Carbon-Credit Note that will serve as a carbon exchange fiat (seal of approval) in carbon markets. The Carbon Credit Note provides a quality benchmark similar in nature to the Certified Organic Seal.

Carbon Registry

Referring back to FIG. 1, the registry 120 brokers carbon credit trading. In some embodiments, the registry 120 is part of the platform. In other embodiments, the registry is a third-party registry. The registry enables the carbon credit market that brings together buyers (entities interested in offsetting their supply chain carbon footprint) and sellers (agricultural producers).

Carbon offset registries track offset projects and issue offset credits for each unit of emission reduction or removal verified and certified. Registries create a credible, fungible offset commodity by recording the ownership of credits. Enforcement systems assure that contracts identify the right of offset credit and define who bears the risk in case of project failure. In some embodiments, a carbon metrics' documentation process assigns a serial number to each verified offset credit. Upon sale, this serial number is transferred from the seller to the buyer's carbon metrics account.

A buyer “uses” the carbon credit by claiming it as an offset against their supply chain carbon emissions. In that case, the registry retires the serial number from the open market. In this manner, registries reduce the risk of double counting (having multiple stakeholders take credit for the same offset.) In some embodiments, a registration and enforcement system can include a carbon registry with publicly available information to uniquely identify carbon offset projects available for sale. A registration and enforcement system can also establish serial numbers for each offset credit generated by each carbon sequestration project in the market.

A registration and enforcement system may also provide a system to transparently track ownership of offsets to make it possible to trace each credit back to the project from which it originated. A registration and enforcement system may still further include a system to quickly check on the status of an offset credit (e.g., whether a carbon credit has been retired) and/or to identity of the carbon credit owner (seller) involved in the transaction. Yet further, a registration and enforcement system can drive carbon credit permanence clauses applicable to the sale and/or provide contractual or legal standards establishing responsibility for project failure or partial project failure (e.g., who is responsible for replacing the credits that the failed project should have produced). Registries can also be set up for voluntary offset markets.

In some embodiments, supply chain emission reductions may use a different carbon market strategy: carbon insets. Carbon insets are not designed to offset the emissions in other parts of the supply chain but rather reduce its overall greenhouse gas emission footprint. A difference between practices that generate carbon offsets and those that create carbon insets is their permanence. By way of example, a time horizon can be utilized to define permanence: e.g., negotiated periods represent carbon offsets contracts. Another difference is that while an agriculture carbon credit can only offset one ton of carbon emitted somewhere else, multiple supply chain stakeholders can claim portions of a carbon inset.

Miscellaneous

Aspects herein provide modeling and simulation environments that permit the assessment of carbon sequestration permanence and leakage in a rigorous manner.

Measure Capture of Carbon

To facilitate the effectiveness of the platform, an aspect herein lies in the ability to better measure the carbon captured in a specific region, e.g., a farmland, field, etc.

Integrating a UAS sensing layer and traditional remote sensing platforms offers a unique opportunity to quantify the carbon sequestration holdings. When combined with machine learning/artificial intelligence models for soil, climate, and agronomic practices, a carbon-calibrated data layer derived using the platform herein, will permit the platform to address the remaining carbon sequestration parameters: permanence and leakage.

The validation and verification methodology herein are a minimal intrusive verification platform, minimizing access to only farming practices associated with climate-smart conservation activities. Moreover, the platform, the carbon capture modeling, the verification/validation assessments, etc., provide a trusted benchmark that companies interested in offsetting their carbon footprint could depend on for climate-smart investment.

Computer System Overview

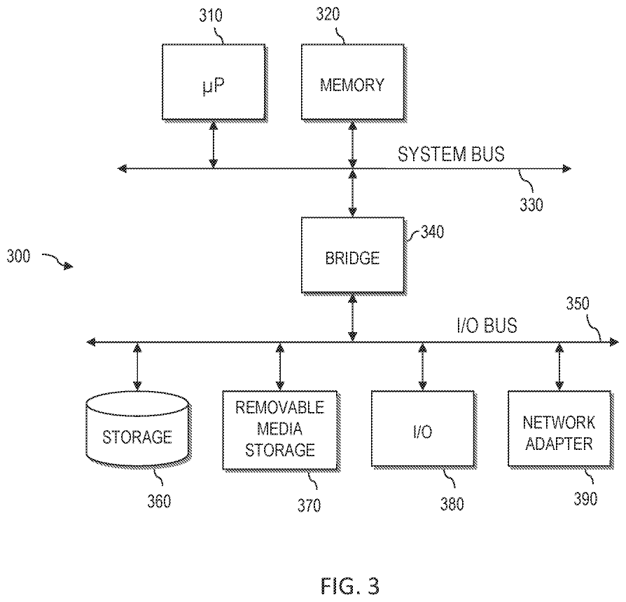

Referring to FIG. 3, a schematic block diagram illustrates an exemplary processing system 300 for implementing the various processes described herein. The exemplary processing system 300 includes one or more (hardware) microprocessors (μP) 310 and corresponding (hardware) memory 320 (e.g., random access memory and/or read only memory) that are connected to a system bus 330. Information can be passed between the system bus 330 (via a suitable bridge 340) and a local bus 350 that is used to communicate with various input/output devices. For instance, the local bus 350 is used to interface peripherals with the one or more microprocessors (μP) 310, such as storage 360 (e.g., hard disk drives); removable media storage devices 370 (e.g., flash drives, DVD-ROM drives, CD-ROM drives, floppy drives, etc.); I/O devices 380 such as input device (e.g., mouse, keyboard, scanner, etc.) output devices (e.g., monitor, printer, etc.); and a network adapter 390. The above list of peripherals is presented by way of illustration and is not intended to be limiting. Other peripheral devices may be suitably integrated into the processing system 300.

The microprocessor(s) 310 control operation of the exemplary processing system 300. Moreover, one or more of the microprocessors(s) 310 execute computer readable code (e.g., stored in the memory 320, storage 360, removable media insertable into the removable media storage 370 or combinations thereof, collectively or individually referred to as computer-program products) that instructs the microprocessor(s) 310 to implement the computer-implemented processes herein.

The computer-implemented processes herein may be implemented as a machine-executable process executed on a computer system, e.g., one or more of the processing devices 102, 112, 118, etc., of FIG. 1; the process 200 of FIG. 2, etc.

Thus, the exemplary computer system or components thereof can implement processes and/or computer-implemented processes stored on one or more computer-readable storage devices as set out in greater detail herein. Other computer configurations may also implement the processes and/or computer-implemented processes stored on one or more computer-readable storage devices as set out in greater detail herein, e.g., with reference to any combination of features described with reference to the any combination of the preceding FIGURES.

Further Example Carbon Offset Platform

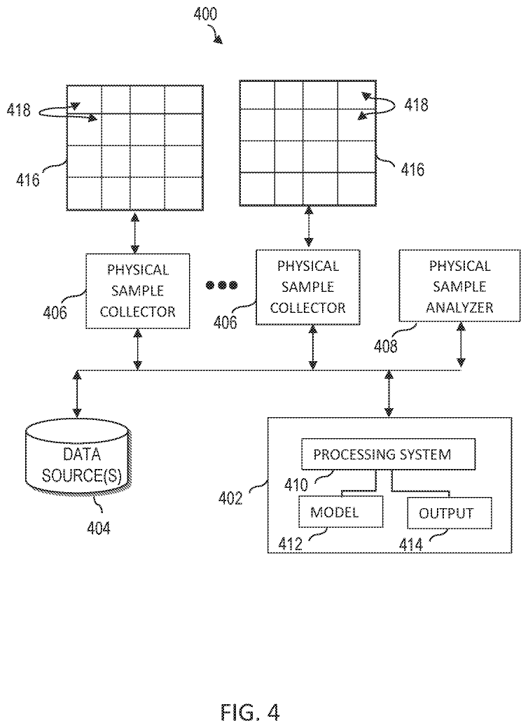

Referring to FIG. 4, an example carbon offset system 400 is provided. The carbon offset system 400 can use any combination of features described with reference to FIG. 1, FIG. 2, FIG. 3 or any combination features in any combination of the previous FIGURES.

As illustrated, the carbon offset system 400 includes generally, a carbon offset platform 402, one or more data sources 404, one or more physical sample collectors 406, and one or more physical sample analyzers 108.

In the example embodiment the carbon offset platform 402 can be implemented by the analysis engine 114 (FIG. 1) interacting with the data sources 116 (FIG. 1) and optionally, any combination of other resources described with reference to FIG. 1, the process 200 (FIG. 2), the processing system 300 (FIG. 3), or any combination thereof.

The carbon offset platform 402 interacts with a data source 404, e.g., a database that stores electronic geospatial-specific data.

Further, the carbon offset platform includes a processing system 410 that measures carbon sequestration using a carbon model 412. As described more fully herein, in some embodiments, the model 412 factors additionality, permanence, leakage, or a combination thereof.

As will be described in greater detail below, the processing system 410 can also generate outputs at 414.

The processing system 410 is further operatively programmed to receive and process at least one sample collected from within a bounded geographic region 416 to determine the amount of carbon sequestered thereby. Here, the distinct, bounded geographic regions 416 are defined by the electronic geospatial-specific data stored in the database of the data source 404.

The samples can be collected using any of the sampling techniques described with reference to sampling at 204 (FIG. 2).

By way of illustration, and not by way of limitation, as shown, a physical sample collector 406 is utilized to collect at least one physical sample from each distinct, bounded geographic region 416. Each collected sample is evaluated by the physical sample analyzer 408. In practice, the physical sample collector 406 and the physical sample analyzer 408 can be integrated into a common device, or the physical sample collector 406 and the physical sample analyzer 408 can be separate devices/systems. Likewise, the physical sample analyzer 408 can be integrated into the processing system 410, or the physical sample analyzer 408 can be a separate process, e.g., an external lab that sends data to the processing system 410.

Moreover, analogous to that described with reference to FIG. 2, the physical sample collector 406 can carry out sampling of actual (physical) samples that are collected from a specified location. As an illustrative example, using remote sensing image data, a geographical area 416 can be parsed into adjacent cells (or regions) 418. In FIG. 4, not every cell 418 is labeled for clarity of illustration. One or more samples can be collected from the associated bounded region, e.g., one or more samples per cell 418. In some embodiments, one or more samples (statistically significant sample number determined based upon application) can be collected from one or more cells 418. In some embodiments, samples from every cell 418 are not strictly required. Rather, in some embodiments, samples can be collected from some, but not necessarily all of the cells 418.

The collected samples are analyzed, e.g., by the physical sample analyzer 408 to determine the amount of carbon sequestered thereby (or a suitable sample feature proxy than can be used to estimate the carbon sequestered at the sample location). For instance, spectral analysis can be implemented to identify the frequency response of the sample. Further, the results of the spectral analysis can be compared against calibrated carbon sequestration stock signature data, e.g., to detect emissions within a predetermined frequency and spectral bandwidth.

In some embodiments, sampling can be implemented by physical collection, which is sent to a laboratory for analysis. The laboratory can be co-located or separately located from the processing system 410. In some embodiments, sampling may be carried out using sensors placed at the location of interest. In some embodiments, the sensors are “smart sensors” that define a processing device 102 (see processing devices 102, which are illustrated directly below the farm fields in FIG. 1) capable of communicating across the network 104 back to the server 112 (FIG. 1).

In some embodiments, the sample collection process is periodic, e.g., yearly, bi-annually, three times a year, or any other timeframe determined to be relevant to the platform. The frequency of the collections will be determined by the agronomic life-cycle with a predetermined minimum number of collection events.

The processing system 410 is also operatively configured to evaluate the collected and processed sample data, a reference baseline, the stored electronic geospatial-specific data, and the model to derive a carbon sequestration stock signature to carbon credit translation, and provide an output.

For instance, as noted more fully herein, the reference baseline indicates a base carbon sequestration for an associated bounded geographic region 416. The sample data for the associated bounded geographic region 416, and data describing the associated bounded geographic region 416 (e.g., number, size, etc., of the cells 418) are used to compute a total carbon sequestration for the associated bounded geographic region 416. As such, the reference baseline can be subtracted from the computed total carbon sequestration to derive an increase in carbon sequestration for the associated bounded geographic region 416.

The model applies additional layers of refinement to the increase in carbon sequestration for the associated bounded geographic region 416, taking into account additionality, permanence, leakage, or a combination thereof.

The above-described example carbon offset system 400 can be modified by any one or more of the below-described embodiments, in any combination.

In some embodiments, the electronic geospatial-specific data is extracted from at least one of remote sensing satellite(s), unmanned aerial systems (UAS), imagery collected from one or more satellites, or third-party servers that have previously collected such geospatial-specific data.

Likewise, in some embodiments, the electronic geospatial-specific data comprises terrestrial frequency responses of agricultural areas.

Also, in some embodiments, the carbon sequestration model uses additionality, permanence, and leakage attributes.

By way of example, in some embodiments, the additionality attribute characterizes the greenhouse gas mitigation that would not have occurred without the acquisition of a carbon offset.

As another example, in some embodiments, the permanence attribute characterizes ensuring the removal of carbon dioxide from the atmosphere is permanent and not reversed at a future point in time. As yet another example, in some embodiments, the permanence attribute characterizes ensuring the removal of carbon dioxide from the atmosphere for a predetermined duration of time, e.g., the duration is at least 100 years.

As still another example, in some embodiments, the leakage attribute characterizes an increase in greenhouse gas emissions outside of a project area in response to decreases in production within the project area.

Yet further, in some embodiments, the processing system is operatively programmed to provide the output as at least one of carbon offset data, carbon credits, carbon sequestration amount, carbon offset amount, carbon sequestration verification, carbon sequestration validation, carbon offset audit, carbon sequestration valuation, or carbon sequestration education.

Also, in some embodiments, the processing system is further configured to establish the reference baseline. As an example, in some embodiments, a reference (carbon sequestration stocks) baseline is derived from source data comprising historical geospatial geo-rectified remote sensing image data of areas of land. Also, in some embodiments, the reference baseline is derived from geospatial-based data comprising remote sensing satellite (or UAS) imagery, which collects images or other relevant data of agricultural areas. As a further example, in some embodiments, the reference baseline is derived in part, from remote sensing geo-rectified geospatial image data that is processed to identify bounded regions that can be attributed to a single source of carbon sequestration pool credit. Still further, in some embodiments, the reference baseline quantifies a soil's organic carbon sequestration content at any given location and time.

Additionally, in some embodiments, at least one sample is analyzed using spectral analysis to evaluate select frequencies or frequency range(s) of the analyzed sample.

In some embodiments, the samples are collected during at least one of: the peak photosynthesis response cycle, the end of a harvesting period, which will coincide with a cover crops planting period, or at an early stage of a planting season. Here, these three sets of carbon in the soil measurements permit the complete characterization of the nature of the carbon sequestration cycle at a given location (carbon sequestration stock signature).

Yet further, in some embodiments, the platform further collects and stores in the database, agronomic data that comprises parameterized inquiries, collected facts, collected extrinsic information, effects on or caused by neighboring regions, historical information, or combinations thereof.

Also, in some embodiments, the evaluation factors distinctive frequency responses that are associated with carbon sequestered in soil.

Additionally, in some embodiment, the output comprises a carbon sequestration stock signature estimation that translates into a trusted carbon credit.

Carbon Sequestered by Forests

The analysis of carbon sequestration, e.g., which may be focused on soils, can be extended to include the determination of carbon collected by forests, or other large areas of vegetation, e.g., green spaces, agricultural areas, plant-rich areas, etc. By way of example, forests, as major carbon sinks, can play a crucial role in the global carbon cycle. Forests absorb carbon dioxide from the atmosphere during photosynthesis and store the absorbed carbon dioxide as biomass. This process, known as carbon sequestration, is a key factor in mitigating climate change. The amount of carbon sequestered by a forest can vary significantly depending on the type of forest, its age, its health, and the climate in which it is located. Therefore, it is essential to accurately measure and monitor the carbon stocks of forests to understand their role in the global carbon cycle and to inform forest management and conservation strategies.

To achieve this, aspects herein can utilize remote sensing data collections from a variety of satellite payloads, including but not limited to Landsat, Aqua, and EOS (Earth Observing System). These satellites provide valuable data that can be used to estimate forest carbon stocks at a large scale. In this regard, aspects herein model the carbon sequestration stock signature returns from these satellites, which provides information about forest structure and biomass. By comparing these model results with a calculated carbon reference baseline (as described more fully herein), the platform estimates the amount of carbon sequestered by the forests. This approach allows the platform to monitor changes in forest carbon stocks over time and across different geographical areas, providing valuable insights for forest management and climate change mitigation strategies.

Notably, the carbon sequestration collection, analysis, and production described herein translates to the estimation of carbon sequestration associated with forests.

### MISCELLANEOUS

According to aspects herein, the process of solutioning involves the collection, analysis, modeling, and production of relevant data. Moreover, aspects herein provide the ability to translate the imagery, collected sample data (e.g., soil data), additional information such as climate information, and conservation practices into a credible estimation of carbon stocks.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The description of the present disclosure has been presented for purposes of illustration and description but is not intended to be exhaustive or limited to the invention in the form disclosed. Many modifications and variations will be apparent to those of ordinary skill in the art without departing from the scope and spirit of the disclosure.

Having thus described the invention of the present application in detail and by reference to embodiments thereof, it will be apparent that modifications and variations are possible without departing from the scope of the invention defined in the appended claims.

What is claimed is:

1. A carbon offset platform comprising:
a database that stores electronic geospatial-specific data, wherein distinct bounded geographic regions can be associated with the stored electronic geospatial-specific data; and
a processing system that measures carbon sequestration using a carbon model that factors additionality, permanence, leakage, or a combination thereof, wherein the processing system is further operatively programmed to:
receive and process at least one physical sample collected from within the bounded geographic region to determine the amount of carbon sequestered thereby;
evaluate the collected and processed sample data, a reference baseline, the stored electronic geospatial-specific data, and the model to derive a carbon sequestration stock signature to carbon credit translation; and
provide an output.

2. The carbon offset platform of claim 1, wherein the electronic geospatial-specific data is extracted from at least one of:
remote sensing satellite(s);
unmanned aerial systems (UAS);
geo-spatial imagery collected from one or more satellites; or
third party servers that have previously collected such geospatial-specific data.

3. The carbon offset platform of claim 1, wherein the electronic geospatial-specific data comprises terrestrial frequency responses of agricultural areas.

4. The carbon offset platform of claim 1, wherein the carbon model uses additionality, permanence, and leakage attributes.

5. The carbon offset platform of claim 4, wherein the additionality attribute characterizes the greenhouse gas mitigation that would not have occurred without the acquisition of a carbon offset.

6. The carbon offset platform of claim 4, wherein the permanence attribute characterizes ensuring the removal of carbon dioxide from the atmosphere is permanent and not reversed at a future point in time.

7. The carbon offset platform of claim 4, wherein the permanence attribute characterizes ensuring the removal of carbon dioxide from the atmosphere for a predetermined duration of time.

8. The carbon offset platform of claim 7, wherein the duration is at least 100 years.

9. The carbon offset platform of claim 4, wherein the leakage attribute characterizes an increase in greenhouse gas emissions outside of a project area in response to decreases in production within the project area.

10. The carbon offset platform of claim 1, wherein the processing system is operatively programmed to provide the output as at least one of:
carbon offset data;
carbon credits;
carbon sequestration amount;
carbon offset amount;
carbon sequestration verification;
carbon sequestration validation;
carbon offset audit;
carbon sequestration valuation; or
carbon sequestration education.

11. The carbon offset platform of claim 1, wherein the processing system is further configured to establish the reference baseline.

12. The carbon offset platform of claim 11, wherein the reference baseline is derived from source data comprising historical geospatial geo-rectified remote sensing image data of areas of land.

13. The carbon offset platform of claim 11, wherein the reference baseline is derived from geospatial-based data comprising remote sensing satellite (or UAS) imagery, which collect images of agricultural areas.

14. The carbon offset platform of claim 11, wherein the reference baseline is derived in part, from remote sensing geo-rectified geospatial image data that is processed to identify bounded regions that can be attributed to a single source of carbon sequestration pool credit.

15. The carbon offset platform of claim 11, wherein the reference baseline quantifies a soil's organic carbon sequestration content at any given location and time.

16. The carbon offset platform of claim 1, wherein the at least one sample is analyzed using spectral analysis to evaluate select frequencies or frequency range(s) of each analyzed sample.

17. The carbon offset platform of claim 1, wherein the at least one sample is collected:
at the peak photosynthesis response cycle;
the end of a harvesting period, which will coincide with a cover crops planting period; and
at an early stage of a planting season; wherein these three sets of carbon in the soil measurements permit the complete characterization of the nature of the carbon sequestration cycle at a given location.

18. The carbon offset platform of claim 1, wherein the platform further collects and stores in the database, agronomic data that comprises parameterized inquiries, collected facts, collected extrinsic information, effects on or caused by neighboring regions, historical information, or combinations thereof.

19. The carbon offset platform of claim 1, wherein the evaluation factors distinctive frequency responses that are associated with carbon sequestered in soil.

20. The carbon offset platform of claim 1, wherein the output comprises a carbon sequestration stock signature estimation that translates into a trusted carbon credit.
